(19) United States
(12) Patent Application Publication
(10) Pub. No.: US 2017/0322221 A1
(43) Pub. Date: Nov. 9, 2017

(54) PREDICTION MARKER

(71) Applicant: TOHOKU UNIVERSITY, Miyagi (JP)

(72) Inventors: Noriko OSUMI, Miyagi (JP); Kaichi YOSHIZAKI, Miyagi (JP)

(73) Assignee: TOHOKU UNIVERSITY, Miyagi (JP)

(21) Appl. No.: 15/527,565

(22) PCT Filed: Nov. 17, 2015
(86) PCT No.: PCT/JP2015/082321

§ 371 Date: May 17, 2017

(30) Foreign Application Priority Data
Nov. 18, 2014 (JP) 2014-233823

(51) Int. Cl. G01N 33/68; C07K 14/47

(52) U.S. Cl. CPC G01N 33/68; C07K 14/47

(57) ABSTRACT

An object of the present invention is to provide a prediction marker in male animals for predicting behaviors of their offspring. Specifically, isolated H3K79me3 from a spermatocyte or a sperm of a male animal is used as a marker. When levels of methylation of H3K79me3 in a spermatocyte or a sperm which has been collected from male animals are analyzed, those with a lower level of trimethylation of H3K79me3 have a higher probability of their offspring having autism or exhibiting autism-like behaviors.

### CROSS REFERENCE TO RELATED DOCUMENTS

This application claims the benefit of Japanese Patent Application No. 2014-233823 filed on Nov. 18, 2014, the entire disclosure of which is hereby incorporated by reference.

### TECHNICAL FIELD

The present invention relates to a prediction marker in male animals for predicting behaviors of their offspring in the next generation.

### BACKGROUND ART

Autism spectrum disorders are a group of developmental disorders characterized by qualitative abnormalities in social interaction, qualitative abnormalities in communication, patterns of repetitive behaviors and restricted interests.

Criteria for establishing mouse models of autistic spectrum disorders have been made. For example, three-chamber social approach tests for revealing qualitative impairments in social interaction, ultrasonic vocalization tests for addressing communication disorder, and reversal learning tests using, for example, a Morris water maze for assessing the perseveration have been proposed. Mouse models of autistic spectrum disorders have actually been created based on them (Seikagaku 83(9):841-845, 2011).

The cause of autism spectrum disorders has not been fully elucidated but it is considered to be caused by congenital brain dysfunction. In human, correlation between paternal age at birth of offspring and susceptibility of the offspring to psychiatric disorders including autism spectrum disorders was reported (D'Onofrio B M et al. “Paternal age at childbearing and offspring psychiatric and academic morbidity.” JAMA Psychiatry 2014 vol. 71, p. 432-438; Reichenberg A et al. “Advancing paternal age and autism.” Arch Gen Psychiatry 2006 vol. 63, p. 1026-1032).

### SUMMARY OF THE INVENTION

### Problems to be Solved by the Invention

An object of the present invention is to provide a prediction marker in male animals for predicting behaviors of their offspring in the next generation.

The inventors of the present invention have found that, by analyzing levels of trimethylation of lysine 79 on histone 3, a nuclear protein, in spermatocytes or sperm collected from male animals, those with a higher level of trimethylation had a greater probability of their offspring having autism or exhibiting autism-like behaviors. The inventors thus accomplished the present inventions.

An aspect of the present invention is a prediction marker in a male animal for predicting a behavior of its offspring, the prediction marker being H3K79me3 in a spermatocyte or a sperm which has been isolated from the male animal. The behavior may be an autism spectrum behavior or an autism spectrum-like behavior.

Another aspect of the present invention is a method of predicting a behavior of an offspring of a male animal, the method including the step of determining a level of methylation of a H3K79me3 in a spermatocyte or a sperm which has been collected from the male animal. The behavior may be an autism spectrum behavior or an autism spectrum-like behavior.

### BRIEF DESCRIPTION OF THE DRAWINGS

FIG. 1 shows the correlation between the age of parent mice (male) and the levels of H3K79me3 in an example of the present invention.

FIG. 2 shows the correlation between the levels of H3K79me3 in parent mice (male) and behaviors of pup mice of the next generation in an example of the present invention.

### EMBODIMENTS FOR CARRYING OUT THE INVENTION

Embodiments of the present invention completed based on the aforementioned findings will be described with examples in detail.

Unless otherwise noted in embodiments and examples, all procedures used are according to standard protocols such as M. R. Green & J. Sambrook (Ed.), Molecular cloning, a laboratory manual (4th edition), Cold Spring Harbor Press, Cold Spring Harbor, N.Y. (2012); F. M. Ausubel. R. Brent, R. E. Kingston, D. D. Moore. J. G. Seidman. J. A. Smith. K. Struhl (Ed.). Current Protocols in Molecular Biology. John Wiley & Sons Ltd., with or without modifications or changes. In addition, commercial reagent kits or measurement instruments are used as described in protocols attached thereto, unless otherwise noted.

The objects, features, advantages, and ideas of the present invention are apparent to those skilled in the art from the description of this specification. Furthermore, those skilled in the art can easily reproduce the present invention from the description herein. The embodiments and specific examples described below represent preferable embodiments of the present invention, which are given for the purpose of illustration or explanation. The present invention is not limited thereto. It is obvious to those skilled in the art that various changes and modifications may be made according to the description of the present specification within the spirit and scope of the present invention disclosed herein.

(1) Prediction Marker in Male Animals for Predicting Behaviors of their Offspring in the Next Generation

The prediction marker of the present invention is isolated H3K79me3 from a spermatocyte or a sperm. H3K79me3 is trimethylated histone H3 at lysine 79 and can be easily detected using, for example, an anti-H3K79me3 antibody. Behaviors of the offspring in the next generation can be predicted based on the level of methylation of H3K79me3. The term “level of methylation of H3K79me3” refers to a percentage of trimethylation of lysine 79 on a histone H3 molecule, which may be represented as an absolute value or a value relative to a level of methylation of H3K79me3 in a certain individual.

Examples of the behaviors to be predicted in the offspring include autism spectrum behaviors and autism spectrum-like behaviors.

The male animal is not particularly limited, and may be a vertebrate, a mammal, a rodent or a primate. In particular, the animal is preferably an experimental animal such as a mouse, a rat, a hamster, a monkey, a marmoset and the like, and most preferably, human.

### (2) Method of Predicting, in a Male Animal, Behaviors of its Offspring in the Next Generation

A prediction method according to the present invention is a method of predicting, in a male animal, behaviors of the offspring in the next generation including the step of determining a level of methylation of H3K79me3 in a spermatocyte or a sperm which has been collected from the male animal. The level of methylation obtained is compared with a standard level in standard male animals. If the level of methylation obtained is higher than the standard level, it is judged that the offspring in the next generation is more likely to exhibit autism behaviors or autism spectrum-like behaviors or have an autism spectrum disorder than the offspring of standard male animals. The autism spectrum-like behavior means, when an individual without the autism spectrum disorder takes the same behavior as individuals with the autism spectrum disorder, that behavior.

The standard level in standard male animals can be determined according to an ordinary method of a person skilled in the art. For example, it can be a predetermined standard level or standard range of the level or a level of methylation in a standard sample determined simultaneously in parallel with samples.

For example, when one or more males of the same ages as a target male for prediction are used as standard males, levels of methylation in the males or their average may be determined in advance and used as the standard level(s). Specifically, a certain range (e.g., errors or standard deviations) may be taken from the average and the range may be used as the standard level; a range in which the values of the levels of methylation obtained fall may be used as the standard level; or a range in which a certain percentage (e.g., 80%, 90%. 95%, 99%, etc.) of the total number of animals fall may be used as the standard level. Instead, the standard level as described above can be determined by treating one or more male samples of the same ages simultaneously with a target male sample for prediction and performing measurements under the same condition. If the level of methylation in the target male is higher than the standard level, the offspring of the target male can be judged to be more likely to exhibit autism spectrum behaviors or autism spectrum-like behaviors than those of other males of the same ages.

The standard males may be males whose ages are different from that of the target male. In this case, such standard levels as described above are determined for each age in advance, and thus, based on the age of the males with the standard level of methylation which matches the level in the target male, it is possible to judge the offspring of the males of which age is likely to exhibit autism spectrum behaviors or autism spectrum-like behaviors to the same extent as the offspring of the target male. Furthermore, by determining a percentage that the offspring of the male actually exhibits autism spectrum behaviors or autism spectrum like-behaviors for each age, it is possible, merely by determining the level of methylation in a target male, to estimate the probability that the offspring will exhibit autism spectrum behaviors or autism spectrum like-behaviors.

The method used to measure the level of methylation of H3K79me3 is not particularly limited. It can be determined according to a routine method. For example, the level of methylation in sperm can be measured using an ELISA, cell staining or flow cytometry method as well as Western blotting using an anti-H3K79me3 antibody.

### Example

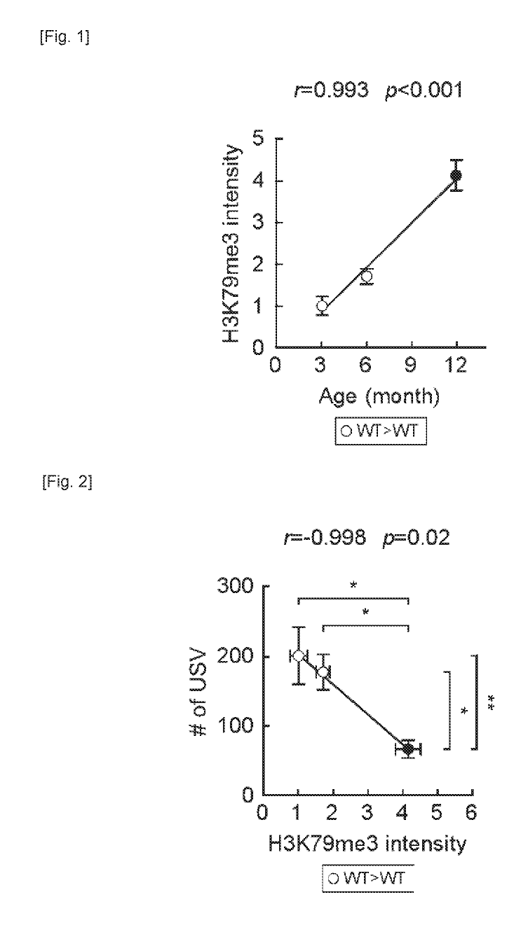

Sperm were collected from male mice (C57BL6/J, three animals of each of 3 months of age, 6-8 months of age, and 12 months of age or older), solubilized in Laemmli sample buffer (containing 1×SDS, 50 mM DTT, 1 mM PMSF, and protease inhibitor cocktail (Roch)) using a sonicator (Microson) (level 10, 5 sec. 3 times) on ice, and then denatured at 95° C. for 10 minutes. Proteins were separated by SDS acrylamide electrophoresis and then Western blotting was performed. First, proteins were transferred to a PVDF membrane (Hybond-P); a rabbit anti-H3K79me3 antibody (Abcam) was used as a primary antibody, and HRP-conjugated anti-rabbit IgG antibody (Millipore) was used as a secondary antibody; ECL Prime Western Blotting Detection Reagent (GE Healthcare Life sciences) was used for detection and signals obtained were captured using a CCD camera. Thereafter, in order to measure a panH3 expression level as an internal control, the antibody was stripped from the membrane with a stripping buffer (100 mM beta-mercaptoethanol. 2% SDS, 62.5 mM Tris-HCl pH 6.8), proteins were treated using a rabbit anti-panH3 antibody (Abcam) as a primary antibody in the same manner as for H3K79me3 to obtain the panH3 expression level. With this panH3 expression level, the level of methylation of H3K79me3 was normalized. Subsequently, with an average level of methylation of the 3-month-old parent mice (male) used as “1,” average relative values of the level of methylation in parent mice (male) of other ages were calculated, which were 1.70 and 4.13 for the parent mice (male) aged 6-8 months and 12 months or older, respectively. Thus, the age of the parent mice (male) and the level of methylation of H3K79me3 were almost proportional (FIG. 1). The correlation coefficient r was 0.993, and the significant difference p was less than 0.001 (t test).

On the other hand, ultrasonic vocalizations of 6-day-old pups descended from parent mice (male) (16 pups from 3 months old mice. 16 pups from 6-8 months old, and 17 pups from 12 months old or older mice) were measured for 5 minutes and averages were each calculated. Specifically, each pup was separated from its mother and brothers and placed in a dish in a sound insulated box. Sound at 25-125 kHz was recorded for 5 minutes using a microphone connected to an UltraSound Gate 416H detector set (Avisoft Bioacoustics) and the number of the times of the sound was calculated.

First, correlation between the level of the methylation of H3K79me3 in the parent mice (male) and the number of the ultrasonic vocalizations made by their offspring was analyzed. As a result, it was found that they were in inverse proportion as shown in FIG. 2, with the correlation coefficient r of −0.998, and the significant difference p of 0.02 (t test). Thus, the number of the ultrasonic vocalizations made by the offspring of the parent mice (male) with the higher level of the methylation of H3K79me3 in their sperm is smaller.

Decrease in number of ultrasonic vocalizations in mice is regarded as a model behavior for autism and therefore the level of methylation of H3K79me3 in a parent mouse (male) can be used as a marker for predicting appearance of autism in its offspring. That is, the offspring of the parent mice (male) with the higher level of methylation of H3K79me3 have higher probability exhibiting autism behaviors or autism-like behaviors.

### INDUSTRIAL APPLICABILITY

The present invention made it possible to provide a prediction marker in male animals for predicting behaviors of their offspring.

1. (canceled)

2. (canceled)

3. A method of predicting a behavior of an offspring of a male animal, the method comprising the step of:
determining a level of methylation of a H3K79me3 in a spermatocyte or a sperm collected from the male animal.

4. The method according to claim 3, wherein the behavior is an autism spectrum behavior or an autism spectrum-like behavior.

5. The method according to claim 3, further comprising predicting a behavior of an offspring of the male animal based on the determined level of methylation of the H3K79me3.

6. The method according to claim 3, further comparing the determined level of methylation of the H3K79me3 with that in a spermatocyte or a sperm collected from a standard male animal.

7. The method according to claim 6, further comprising predicting a behavior of an offspring of the male animal based on the result of the comparison.
